(19) United States
(12) Patent Application Publication
(10) Pub. No.: US 2005/0226825 A1
(43) Pub. Date: Oct. 13, 2005

(54) Topical composition for preventing and treating skin damage caused by UV light exposure

(76) Inventor: Vincent C. Giampapa, Little Falls, NJ (US)

Correspondence Address: MELVIN K. SILVERMAN, 500 WEST CYPRESS CREEK ROAD, SUITE 500, FT. LAUDERDALE, FL, 33309, US

(21) Appl. No.: 11/103,728

(22) Filed: Apr. 11, 2005

Related U.S. Application Data
(60) Provisional application No. 60/561,857, filed on Apr. 13, 2004.

(51) Int. Cl. A61K 7/42; A61K 35/78

(52) U.S. Cl. 424/59; 424/769

(57) ABSTRACT

A topical composition includes a water soluble extract of an Uncaria species in a concentration range from about 0.1% to 15%, and a dermatologically acceptable medium suitable for topical application. The topical composition can further contain deuterium reduced water which has a deuterium concentration in a range from 0.1 ppm to about 110 ppm. Also disclosed are methods using the topical composition for improving skin cell's resistance to DNA damage and/or enhancing skin cell's DNA repair capacity; for protecting the skin from damage, and/or treating skin damage, caused by exposure to ultraviolet light; for treating and/or preventing skin wrinkles, keratosis and lipofuscin deposit.

### CROSS REFERENCE TO RELATED APPLICATION

This application claims the benefit under 35 USC 119 (e) of the provisional patent application Ser. No. 60/561,857, filed on Apr. 13, 2004, which is hereby incorporated by reference in its entirety.

### FIELD OF THE INVENTION

This invention relates to a topical composition and a method of enhancing skin cell resistance to DNA damage and improving DNA repair capacity, protecting skin from damage or treating skin damage, caused by exposure to ultraviolet light, and treating and/or preventing skin wrinkles, keratosis and lipofuscin deposit.

### BACKGROUND OF THE INVENTION

The skin is the most environmentally-stressed organ in mammals, particularly in humans. Not only is the skin subjected to toxic chemicals and hostile environments, but it also is the only organ directly exposed to ultraviolet (“UV”) light in the presence of oxygen. Lengthy exposure of the skin to UV light typically damages the skin, resulting in sunburn, photoaging such as keratosis, lipofuscin deposit, and carcinogenesis. Keratosis is a localized overgrowth of the upper layer of skin. Common forms of keratosis include aging (senile keratosis) and sun exposure (actinic keratosis). Lipofuscin deposit is brownish pigment left over from the breakdown and absorption of damaged blood cells, which is found in smooth muscles, and is also called the “aging” pigment. The effects of ultraviolet radiation from exposure to the sun on human skin are a growing concern for today's longer-lived population.

UV light can be divided into three wavelength regions. UVA is from 400 nm to 320 nm, UVB is from 320 nm to 290 nm, and UVC is from 290 nm to 100 nm. UVC usually does not reach the earth because it is absorbed by the ozone layer. UVA radiation has been shown to penetrate the under layers of the skin, and produce oxidative DNA damage. UVA radiation stimulates the formation of reactive oxygen species, which causes oxidative stress and damaged DNA. UVB radiation, commonly referred to as the sunburn rays, is the one that cause the most concern.

It is well known that following exposure of the skin to ultraviolet radiation free radicals are generated. In the skin, these radicals frequently trigger the release of inflammatory mediators. Several theories have been developed recently in understanding effects of free radicals and inflammation on skin aging.

Giampapa, The Basic Principles and Practice of Anti-Aging Medicine & Age Management, Self-published, Nespitt Granphics Inc., 2003, provides a review on skin aging, with a focus on understanding the oxidative stress and gene expression. More specifically, among the inflammatory chain activities triggered by free radicals, it is known that the transcription factors NF-kB and activator protein 1 (AP-1) are activated by free radicals and pro-inflammatory cytokines, which are generated by free radical activity. NF-kB and AP-1 play critical roles in the regulation of pro-inflammatory cytokines and related proteins, and collagen-digesting enzymes. NF-kB stimulates pro-inflammatory cytokine genes such as IL-1, IL-2, IL-6, IL-8 and TNF-α along with genes that code for cell adhesion molecules and the Cox-2 enzyme. AP-1 mediates induction of genes that express collagen-digesting enzymes (collagenases and metalloproteinases). Because free radicals and pro-inflammatory cytokines activate each of these transcription factors, the result is a self-reinforcing pro-inflammatory cycle. This pro-inflammatory cycle, together with the free radical damage to the cell membrane, produce intracellular free radicals that oxidize the polyunsaturated fatty acids (PUFA) rich membranes surrounding the cytoplasm's organelles, mitochondria and nucleus. It is know that the mitochondria produce cellular energy via the ATP cycle, and most repair and reproduction activities occur via production of proteins by organelles, as directed by the cell's genetic material. Thus, the pro-inflammatory cycle initiated by oxidative stress on the cell membrane gradually weakens the most basic functions of dermal cells, while producing the enzymes that yield collagen damage, microscarring and formation of skin wrinkles.

At the molecular lever, it is known that UVB irradiation produces non-oxidative DNA damage, which results in DNA-structural changes through dimer formation. More specifically, UVB irradiation causes DNA damage in skin by linking adjacent bases to form cyclobutane purimidine dimer (CPD). These may be of the thymine-thymine type, or they may be between adjacent cytosines, or other combinations. CPDs are slowly removed from the DNA by a natural excision repair process, which removes about 50% of the CPDs in 24 hours. Furthermore, it is believed that a combination of UVA and UVB damage may be the formation of CC-TT dimer tandem doubles. These tandems may occur on the p53 tumor suppressor gene and may lead to the formation of squamous cell carcinomas.

It can be appreciated that danger occurs when DNA is damaged but not severely enough to stimulate apoptosis (natural cell death), the damaged cells then reproduce the unrepaired DNA, which initiates a continuum. The skin loses moisture and becomes dull, dry, and rough without tone and texture. Blotches, hyperpigmentation, fine lines and wrinkles develop. Next, premalignant actinic keratoses are formed. Finally these age spots may become malignant squamous cell carcinomas.

The oxidative damage can be assessed by measuring various hydroxy adducts of DNA, such as 8-hydroxyguanine DNA in urine, 8 oxoguanine, Thiamine glycol, 8 hydroxy 2 deoxy guanocine, and doxorubicin induced single strand breaks. Low levels of oxidative products are desirable.

The non-oxidative DNA alteration damage is commonly assessed by quantifying the formation of TT dimers within the cells. By utilizing human living epidermal cell equivalents HaCaT Keratinocytes the amount of cyclobutyl pyrimidine TT dimers (CPD) formed can be measured.

Conventional skin protection efforts typically attempt to either shield the skin from UV light to prevent the production of free radicals, or provide additional agents capable of neutralizing the free radicals. A number of antioxidants have been tested as photoprotective agents, however, results from these studies indicate that the ability of these agents to provide protection is variable.

On the other hand, various pharmaceutical or cosmetic products have been developed for treating age spots resulted from chronic UV light exposure. Zinc peroxide has been utilized in anhydrous ointments as a bleaching agent. Monobenzyl ether of hydroquinone was marketed for its skin lightening effect. Ascorbic acid preparations have been suggested as useful. Niacin and nitroxide have been reported being effective in skin lightening. These products treat age spots by bleaching, and topical color reduction by chemical reactions, but they do not repair skin damages, or prevent re-occurrence of skin pigmentation.

The prior art efforts, either shielding the skin from UV light to prevent the production of free radicals, or neutralizing the free radicals, do not address the inflammatory chain activities in the skin cells caused by the free radicals, repairing the damage at DNA level, or resistance to the DNA damage.

As taught in U.S. Pat. Nos. 6,039,949, 6,238,675, 6,361,805 (to Pero), water soluble extract of the Uncaria species, commercially known as Activar AC-11™, or C-MED-100®, hence its bioactive component, carboxyl alkyl esters, is known to give profound nutritional support as a dietary supplement because the carboxyl alkyl esters enhance both DNA repair and immune cell responsiveness, which, in turn, are the critical physiological processes that regulate aging. Both of these processes involve regulating NF-kB. NF-kB is well known to control (i) the nuclear events that salvage cells from apoptotic cell death and (ii) pro-inflammatory cytokine production. (Beg, et al., An essential role for NF-kB in preventing TNF-α induced cell death Science 274: 782-784, 1996; Wang et al., TNF-α and Cancer Therapy-induced Apoptosis: Potentiation by Inhibition of NF-KB. Science 274: 784-787, 1996).

Pero teaches that the water soluble extract of an Uncaria species and its bioactive component carboxyl alkyl esters, effectively induce apoptosis in HL-60 leukemic cells in vitro, which is the result of NF-kB inhibition. Therefore, the water soluble extract of an Uncaria speciespossesses anti-tumor, anti-inflammatory and immune stimulating properties. Pero further teaches that the water soluble extract of an Uncaria speciesenhances DNA repair in both rats and humans, wherein the DNA repair process removes DNA damages that inhibit cell replication and immune function. Moreover, Pero teaches methods for inhibiting inflammatory response and treating disorders associated with the inflammatory response by inhibiting TNF-α production or induce apoptosis of white blood cells, by orally administering the water soluble extract of the Uncaria species. However, the prior art does not teach the use of water soluble extract of an Uncaria speciesin treating skin damage or deterioration, such as formation of wrinkles and age spots, resulted from chronic UV light exposure.

On the other hand, as taught in U.S. Pat. Nos. 5,855,921 and 5,788,953 (to Somlyai), deuterium reduced water having deuterium concentration from 0.1 ppm to 110 ppm inhibits tumor growth in animal model study. It has also been reported in human clinical trials involving hundreds of cancer patients in Hungary that deuterium reduced water has extended life span of cancer patients. It is believed that deuterium is a component of a submolecular regulating system and the processes temporarily elevating the concentration of deuterium trigger cell proliferation. Somlyai teaches that by administering water or aqueous solutions containing deuterium in an amount less than the deuterium content of natural water, the deuterium level in the human organs can be decreased as a result of exchange processes and in this way the proliferation of tumorigenic cells can be stopped or the development of cancerous tumors can be prevented. However, Somlyai does not teach or recognize whether deuterium reduced water is related to DNA repair process.

It is known that in nature the ratio of hydrogen to deuterium is about 6000:1. Because of the mass difference of 100% the two isotopes show different behavior in chemical reactions. It is a generally accepted view that the D-bonds participating in chemical reactions is split at a lower rate because of the isotope effect, therefore they need an augmented activation energy. In enzymatic reactions it has been measured that the reaction rate is of 4 to 5 times higher with hydrogen than with deuterium.

A world-wide survey of hydrogen isotopes in precipitations revealed that the deuterium content is in a range of 120-160 ppm depending mainly on the locations. According to the measurements of the survey, the deuterium content of the rainfall on the tropics is 155-160 ppm, whereas the deuterium content is only 120-150 ppm in the temperate zones of the world.

It is desirable to provide a topical composition enabling NF-kB inhibition, which in turn, inhibits TNF-α and other inflammatory cytokines IL-1α, IL-β, IL-6 in the skin cells. It is further desirable to enhance the resistance of skin cells to DNA damage by enhancing DNA repair capacity of the skin cells. Moreover, it is also desirable to combine the above-described effect of the water soluble extract of an Uncaria species with deuterium reduced water in a topical composition to provide enhanced protection of the skin cells, and to prevent squamous cell carcinomas.

### SUMMARY OF THE INVENTION

In one embodiment, the present invention is directed to a topical composition which comprises a water soluble extract of an Uncaria species in a concentration range from about 0.1% to about 15% (w/w), and a dermatologically acceptable medium suitable for topical application. Preferably, the water soluble extract of an Uncaria species is in a concentration range from about 0.5% to 8% (w/w).

In a further embodiment, the topical composition further comprises deuterium-reduced water, which has a deuterium concentration in a range from 0.1 ppm to about 110 ppm.

In a further aspect, the present invention is directed to a method of improving skin cell's resistance to DNA damage, and/or enhancing skin cell's DNA repair capacity, which comprises topically applying an effective amount of the topical composition of the present invention on the skin.

In a further embodiment, the present invention is directed a method of protecting the skin from damage caused by exposure to the ultraviolet light, which comprises topically applying an effective amount of the topical composition of the present invention on the skin, prior to exposure to the ultraviolet light, to protect the skin from damage.

In another embodiment, the present invention is directed to a method of treating the skin damage caused by exposure to the ultraviolet light, which comprised topically applying an effective amount of the topical composition of the present invention on an area of the skin, after exposure of the area to the ultraviolet, light to treat the skin damage caused by the ultraviolet light.

In yet a further embodiment, the present invention is directed a method of treating, and/or preventing skin wrinkles, keratosis or lipofuscin deposit, which comprises topically applying an effective amount of the topical composition of the present invention on the skin to reduce or prevent the skin wrinkles, keratosis or lipofuscin deposit.

### BRIEF DESCRIPTION OF THE DRAWINGS

FIG. 1 shows CPD scores at 24 hours after UVB irradiation of human HaCaT (keratinocyte line) cells which are untreated, and treated with 0.5%, 1.5% and 3% of Activar AC-11™ solutions, respectively.

### DETAILED DESCRIPTION OF THE INVENTION

In one embodiment, the present invention provides a topical composition which comprises the water soluble extract of an Uncaria species and a dermatologically acceptable medium suitable for topical application.

The Uncaria species includes tomentosa, guianensis, pteropoda, homomalla, perrottetii, or rhynchopylla. The term “water soluble extract of an Uncaria species” used herein refers to the water soluble extract of an Uncaria species obtained using the method described in U.S. Pat. Nos. 6,361,805, 6,238,675 and 6,039,949, which are hereby incorporated by reference in their entirety. Furthermore, the bioactive active component of the water soluble extract material of the Uncaria species has been identified as carboxy alkyl esters, as described in co-pending patent application Ser. No. 10/093,794, which is hereby incorporated by reference in its entirety. The term of “dermatologically acceptable medium” used herein means the medium that contains one or multiple carrier materials which are dermatologically acceptable. The carrier materials include various chemical compounds, as described in detail hereinafter, which are used to support producing the topic composition in various product forms.

The water soluble extract of an Uncaria species is commercially available under the product name Activar AC-11 ™, or C-Med-100®, from Optigenex, Inc, New York, N.Y. More specifically, Activar AC-11™ (AC-11 hereinafter) is a hot water extract from the bark of Uncaria tomentosa, produced according to the process described in U.S. Pat. No. 6,039,949. Briefly, the extract is produced from heating 150 gm of bark in 5 liters of tap water for 12 hours at 95° C., decanting the soluble fraction, ultra-filtrating the resulting water extract to remove all components having molecular weight larger than 10,000. The fraction having molecular weight less than 10,000 is spray dried. The product is in a form of beige to brown-orange hygroscopic fine powder, and it contains no less than 16% of carboxy alkyl esters, less than 0.05% of indole alkaloids (<10,000 Daltons) and 0% of indole alkaloids (>10,000 Daltons), and it is readily soluble in water (solubility in water >400 mg/ml).

The concentration of the water soluble extract of an Uncaria species in the form of AC-11 in the topical composition is in a range from about 0.1% to about 15% by weight (w/w), preferably from about 0.5% to about 8% (w/w). The topical composition can be produced to have different concentration ranges depending on the utility. For routine use for protection from regular sun exposure, the concentration of the water soluble extract of an Uncaria species can be in the lower portion of the range described above. For protection from severe sun exposure, skin repairing from sun burn, or wrinkle and age spot treatments, the concentration of the water soluble extract of an Uncaria species can be higher.

The topical composition can further include a safe and effective amount of a penetration enhancer. The term of “safe and effective amount” means an amount sufficient to enhance penetration of the water soluble extract of an Uncaria species into the skin, but not to cause side effects or skin reactions. In general, the enhancer can be from about 1% to about 5% (w/w) of the composition. One example of useful penetration enhancers, includes a penetration-enhancing vehicle which comprises (a) N-(2-hydroxyethyl)-pyrrolidone and (b) a cell envelope disordering compound selected from methyl laurate, oleic acid, oleyl alcohol, monoolein, myristyl alcohol, or mixtures thereof. The components (a) and (b) are present in a ratio of (a):(b) of about 1:5 to about 500:1 by weight. A further example is a penetration enhancer that comprises the penetration enhancing agent 1-dodecyl-azacycloheptan-2-one, and a penetration enhancing diol or cycloketo compound. Suitable diol or cycloketo compound includes, but is not limited to, 1,2-propanediol, 1,3-propanediol, 1,2-butanediol, pyrrolidone; 1-(2-hydroxyethyl)azacyclopentan-2-one, and mixtures thereof. Another example of a penetration enhancer comprises about 0.1% (w/w) of a sugar ester, and about 0.1% (w/w) of a phosphine oxide. Suitable sugar ester includes, but is not limited to, sucrose monooctanoate, sucrose monodecanoate, sucrose monolaurate, sucrose monomyristate, sucrose monopalmitate, sucrose monostearate, sucrose monooleate, and sucrose dioleate. The phosphine oxide compound includes, but is not limited to, octyl or monyl or decyl or undecyl or dodecyl-dimethyl phosphine oxide, and the 2-hydroxydecyl derivative thereof. Other delivery vehicles, such as liposome, nanosome or rivosome, can also be used in the topical composition.

The topical composition can also include antioxidants including those well known to those experienced in the art. Representative antioxidants include Vitamin E, tocopheryl acetate, betaglucan, coenzyme Q10, butylated hydroxytoluene (BHT), and superoxide dismutose. Optionally, the topical composition can further include estrogen.

Other conventional skin care product additives can also be included in the compositions of the present invention. For example, collagen, hyaluronic acid and its salts, elastin, hydrolysates, primrose oil, jojoba oil, epidermal growth factor, soybean saponins, mucopolysaccharides, and mixtures thereof may be used.

Additionally, the topical composition can include a wide range of optional ingredients including, buffers, pH adjusting agent; emollients; emulsifying agents; emulsion stabilizers and viscosity builders; humectants; odorants; preservatives, chemical stabilizers; solvents; antifoaming agents; and thickening, stiffening and suspending agents.

Exemplary buffers and pH adjusting agent include ammonium hydroxide, citric acid, diisopropanolamine, hydrochloric acid, lactic acid, monobasic sodium phosphate, sodium citrate, sodium hydroxide, sodium phosphate, triethanolamine, and trolamine.

Exemplary emollients include caprylic/capric triglyerides, castor oil, ceteareth-20, ceteareth-30, cetearyl alcohol, ceteth 20, cetostearyl alcohol, cetyl alcohol, cetyl stearyl alcohol, cocoa butter, diisopropyl adipate, glycerin, gyceryl monooleate, glyceryl monostearate, glyceryl stearate, isopropyl myristate, isopropyl palmitate, lanolin, lanolin alcohol, hydrogenated lanolin, liquid paraffins, linoleic acid, mineral oil, oleic acid, white petrolatum, polyethylene glycol, polyoxyethylene glycol fatty alcohol ethers, polyoxypropylene 15-stearyl ether, propylene glycol stearate, squalane, steareth-2 or -100, stearic acid, stearyl alcohol and urea. As used herein, “emollients” refer to materials used for the prevention or relief of dryness, as well as for the protection of the skin. A wide variety of suitable emollients are known and may be used herein. Sagarin, Cosmetics, Science and Technology, 2nd Edition, Vol. 1, pp., 32-43 (1972), incorporated herein by reference, contains numerous examples of suitable materials. Particularly useful emollients which provide skin conditioning are glycerol, hexanetriol, butanetriol, lactic acid and its salts, urea, pyrrolidone carboxylic acid and its salts, amino acids, guanidine, diglycerol and triglycerol.

Exemplary emulsifying agents include aluminum starch octenylsuccinate, ammonium hydroxide, amphoteric-9, beeswax, synthetic beeswax, carbomer 934, carbomer 934P, carbomer 940, ceteareth-20, ceteareth-30, cetearyl alcohol, ceteth 20, cetyl alcohol, cholesterol, cyclomethicone, diglycerides, dimethicone (e.g., dimethicone 350), disodium monooleamidosulfosuccinate, NF emulsifying wax, fatty acid pentaerythritol ester, glycerides, glyceryl monooleate, glyceryl monostearate, lanolin, lanolin alcohol, hydrogenated lanolin, magnesium stearate, mineral oil, monoglycerides, polyethylene glycol, PEG 100 stearate, polyethylene glycol 6000 distearate, polyethylene glycol 1000 monocetyl ether, polyethylene glycol monostearate, polyethylene glycol 400 monostearate, polyoxyethylene glycol fatty alcohol ethers, polyoxyl 20 cetostearyl ether, polyoxyl 40 stearate, polysorbate 20, polysorbate 40, polysorbate 60, polysorbate 80, polysorbates, PPG-26 oleate, propylene glycol stearate, quaternium-15, simethicone, sodium laureth sulfate, sodium lauryl sulfate, sorbitan esters, sorbitan monolaurate, sorbitan monooleate, sorbitan monopalmitate, sorbitan monostearate, sorbitan palmitate, sorbitan sesquioleate, steareth-2, steareth-100, stearic acid, stearyl alcohol, triethanolamine and trolamine.

Exemplary emulsion stabilizers and viscosity builders include carbomer 934, carbomer 934P, carbomer 940, cetearyl alcohol, cetostearyl alcohol, cetyl alcohol, cetyl stearyl alcohol, dextrin, diglycerides, disodium edetate, edetate disodium, glycerides, glyceryl monostearate, glyceryl stearate, hydroxypropyl cellulose, monoglycerides, plasticized hydrocarbon gel, polyethylene glycol 300, polyethylene glycol 400, polyethylene glycol 1450, polyethylene glycol 8000, polyethylene glycols, propylene glycol stearate and stearyl alcohol.

Exemplary humectants include glycerine, propylene glycol, sorbitol and urea. Exemplary odorants include hypoallergenic perfume, and menthol.

Exemplary preservatives and chemical stabilizers for stability of the composition include alcohol, benzyl alcohol, butylated hydroxyanisole, butylated hydroxytoluene, butylparaben, calcium acetate, caster oil, chlorocresol, 4chloro-m-cresol, citric acid, disodium edetate, Dowicil 200 (Dow), edetate disodium, ethoxylated alcohol, ethyl alcohol, glycerin, Glydant Plus (Lonza), 1,2,6-hexanetriol, Kathon CG (Rohm & Haas), Liquid Germall Plus (ISP Sutton Labs), Liquipar (ISP Sutton Labs), methylparaben, parabens, potassium sorbate, propyl gallate, propylene glycol, propylparaben, sodium bisulfite, sodium citrate, sodium metabisulfite, sorbic acid, tannic acid, triglycerides of saturated fatty acids, Ucarcide (Union Carbide), and zinc stearate.

Exemplary solvents include alcohol, castor oil, diisopropyl adipate, ethoxylated alcohol, ethyl alcohol, fatty alcohol citrate, glycerin, 1,2,6-hexanetriol, hexylene glycol, isopropyl alcohol, isopropyl myristate, isopropyl palmitate, mineral oil, phosphoric acid, polyethylene glycol 300, polyethylene glycol 400, polyethylene glycol 1450, polyethylene glycol 8000, polyethylene glycol 1000 monocetyl ether, polyethylene glycol monostearate, polyethylene glycol 400 monostearate, polyethylene glycols, polyoxyl 20 cetostearyl ether, polyoxypropylene 15-stearyl ether, polysorbate 20, polysorbate 40, polysorbate 60, polysorbate 80, polysorbates, propylene carbonate, propylene glycol, purified water, and SD alcohol 40, triglycerides of saturated fatty acids.

Exemplary antifoarming agents include cyclomethicone, dimethicone (e.g., dimethicone 350) and simethicone.

Exemplary thickening, stiffening and suspending agents include aluminum stearate, beeswax, synthetic beeswax, carbomer 934, carbomer 934P, carbomer 940, cetostearyl alcohol, cetyl alcohol, cetyl esters wax, dextrin, glyceryl monostearate, hydroxypropyl cellulose, kaolin, paraffin, petrolatum, polyethylene, propylene glycol stearate, starch, stearyl alcohol, wax, white wax, xanthan gum, and bentonite.

The topical composition of the present invention can be provided in a wide variety of forms such as liquid, lotions, creams, sprays, sticks, gels, ointments, pastes, and cosmetics.

Lotions and creams can be formulated as emulsions. Typically such lotions comprise from about 0.2% to about 8%, preferably from about 0.5% to about 5%, of the water soluble extract of the Uncaria species; from about 1% to about 20%, preferably from about 5% to about 10%, of one or more emollients; from about 25% to about 95%, preferably from about 45% to about 90%, water; and from about 1% to about 10%, preferably from about 2% to about 5%, of emulsifier. Creams typically comprise from about 0.5% to about 8%, preferably from about 2% to about 5%, of the water soluble extract of the Uncaria species; from about 1% to about 20%, preferably from about 5% to about 10%, of one or more emollients; from about 20% to about 80%, preferably from about 30% to about 70%, water; and from about 1% to about 10%, preferably from about 2% to about 5%, of one or more emulsifiers.

When the topical composition is formulated as a gel or a cosmetic stick, a suitable amount of a cosmetic thickening agent as described above can be used.

In a further embodiment, the present invention provides a topical composition which comprises the water soluble extract of the Uncaria species, deuterium-reduced water, and dermatologically acceptable carrier materials. The dermatologically acceptable carrier materials have been described above.

The term “deuterium-reduced water” used herein means an aqueous fluid having a deuterium concentration substantially below a naturally occurring deuterium level in water, more specifically, having a deuterium level in a range from about 0.1 ppm to about 110 ppm.

The deuterium-reduced water can be produced by distillation or electrolysis, as described in U.S. Pat. Nos. 5,855,921 and 5,788,953, which are hereby incorporated by reference in their entirety. Using electrolysis, the deuterium concentration of water can be reduced down to 30-40 ppm and further reduced to 6-20 ppm by a further electrolysis. Using distillation, the deuterium concentration of water can be reduced down to 20-30 ppm and further reduced to 1-10 ppm by further increasing the plate number and/or repeating the distillation process. To produce a large volume of water, this deuterium depleted water can be mixed with regular water in a predetermined proportion to obtain a water which has the deuterium concentration from about 80 to about 110 ppm, which is substantially lower than the natural occurring level of deuterium in water.

In a further aspect, the present invention provides a method for protecting skin from damage, or treating skin damage, caused by exposure to ultraviolet light, for treating and preventing skin wrinkles, and for treating and preventing keratosis and lipofuscin. The method comprises topically applying an effective amount of the topical compositions of the present invention, as described above, on the skin. For protection from regular sunlight exposure, the topical composition can be applied on a daily basis on the areas of body, which have frequent sun exposure, such as face, neck and hands, particularly prior to expose to the sunlight. For treating the skin damage caused by exposure to ultraviolet light, the topical composition can be applied to the exposed areas after the exposure.

The water soluble extract material of an Uncaria species of the topical composition of the present invention inhibits NF-kB, which in turn inhibits the stimulation of TNF-α and the other inflammatory cytokines IL-1α, IL-β and IL-6. Consequently, the inflammatory chain activities triggered by the free radicals are hindered, which prevents microscarring and the formation of skin wrinkles. Furthermore, the water soluble extract material of an Uncaria species enhances DNA repair capacity of fibroblast, which enhances the resistance of the skin cells to DNA damage caused by the inflammatory activities, and promotes production of collagen. Moreover, as illustrated hereinafter in detail, the water soluble extract material of an Uncaria species enhances DNA repair capacity of the skill cells to the non-oxidative damages caused by exposure to UVB. Because the water soluble extract material of an Uncaria species prevent and/or treat both oxidative and non-oxidative damages resulted from exposure to ultraviolet light, it is far more effective than a simple anti-oxidant in repairing/preventing the broad spectrum of DNA damages.

On the other hand, the deuterium-reduced water reduces D₂O concentration in cellular environment. Since hydrogen bonds formed with D2O is stronger than that formed with H₂O, reducing concentration of D2O in cellular environment is expected to reduce the activation energy required in unwinding the DNA helix, hence facilitate DNA repairing process. In combination, the water soluble extract material of an Uncaria species and deuterium-reduced water synergistically improve the resistance of the skin cells to DNA damage, enhance DNA repair capacity, and prevent wrinkle, keratosis and lipofuscin deposit.

Example 1 illustrates an example of determining the effect of AC-11 on repairing CPDs. Human HaCaT keratinocyte cells were treated with three test solutions having 0.5%, 1.5% and 3% of AC-11 and irradiated with UVB. DNA damage persisting at 24 h was assayed by immunofluorescence. The results show that cells incubated with AC-11 solutions before and after exposure to UVB have 16-25% fewer CPDs than untreated cells. This shows that AC-11 improves cell's resistance to DNA damage and enhances DNA repair capacity, as reflected by less amount of dimer formation after UVB exposure.

The following example further describes and demonstrates embodiments within the scope of the present invention. The examples are given solely for the purpose of illustration and are not to be construed as limitations of the present invention, as many variations thereof are possible without departing from the spirit and scope of the invention.

### EXAMPLE 1

Methods

The repair of cyclobutane purimidine dimers (CPDS) was measured in cell culture over 24 hours using antibodies against CPD. Human HaCaT (keratinocyte line) cells were seeded on glass slides, and pretreated for 24 hours with three testing solutions containing 0.5%, 1.5% and 3% of AC-11. Two samples were untreated and used as control. The cells were then irradiated with 500 J/m² UVB (Kodacel filtered FS40 sunlamp). One treated sample was fixed immediately for assay. The remaining samples were incubated with the appropriate test solutions for 24 hours. At that point, all samples were fixed and stained with antibodies specific for CPD in DNA. The binding was visualized by fluorescent secondary antibody and fluorescence microscopy. CPDs were measured by a semi-quantitative scoring system. Statistical analysis was performed by using ANOVA (Instat, GraphPad).

Results

The measurement results of CPD are expressed as CPD score as shown in Table 1.

TABLE 1
CPD Score

| | | Incubation | CPD | Standard | Number of |
|---|---|---|---|---|---|
| UV | Treatment | Time (h) | score | Deviation | Observations |
| None | None | 0 | 0.58 | 0.50 | 50 |
| 500 J/m² | None | 0 | 3.38 | 0.61 | 100 |
| 500 J/m² | None | 24 | 3.55 | 0.64 | 100 |
| 500 J/m² | AC-11, 0.5% | 0 | 2.84 | 0.68 | 50 |
| | (5 mg/ml) | | | | |
| 500 J/m² | AC-11, 0.5% | 24 | 2.81 | 0.92 | 100 |
| | (5 mg/ml) | | | | |
| 500 J/m² | AC-11, 1.5% | 0 | 3.76 | 0.43 | 50 |
| | (15 mg/ml) | | | | |
| 500 J/m² | AC-11, 1.5% | 24 | 2.66 | 0.82 | 50 |
| | (15 mg/ml) | | | | |
| 500 J/m² | AC-11, 3.0% | 0 | 3.08 | 0.81 | 100 |
| | (30 mg/ml) | | | | |
| 500 J/m² | AC-11, 3.0% | 24 | 2.98 | 0.60 | 100 |
| | (30 mg/ml) | | | | |

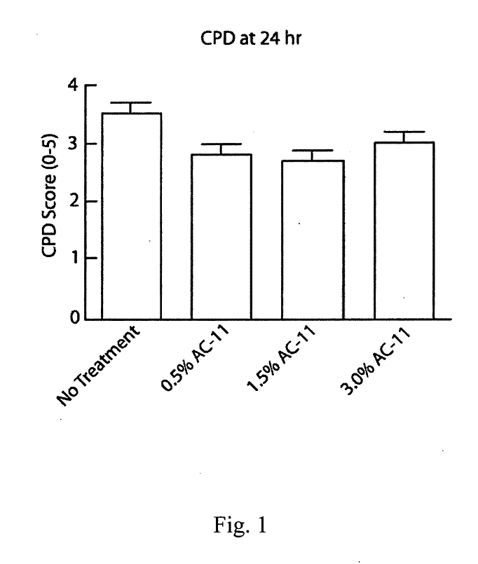

The CPD scores at 24 hours of the untreated and treated samples are shown graphically in FIG. 1. It is noted that the error bars in FIG. 1 are standard error of the mean. The percent difference was calculated as the difference in CPD score between “no treatment” and treatment scores, divided by the “no treatment” score. The raw data were analyzed for statistical significance using the Kruskal-Wallis Test. The results are shown in Table 2.

TABLE 2
Results of Kruskal-Wallis Test

| Comparison | Percent Difference | p-value |
|---|---|---|
| No treatment vs 0.5% AC-11 | 21% | p < 0.001 |
| No treatment vs 1.5% AC-11 | 25% | p < 0.001 |
| No treatment vs 3.0% AC-11 | 16% | p < 0.001 |

The results showed that cells incubated with AC-11 solutions before and after exposure to UVB showed 16-25% fewer CPDs than untreated cells. This demonstrates that AC-11 improves cell's resistance to DNA damage and enhances DNA repair capacity, as reflected by less amount of dimer formation after UVB exposure.

While the invention has been disclosed in connection with certain preferred embodiments, this should not be taken as a limitation to all of the provided details. Modifications and variations of the described embodiments may be made without departing from the spirit and scope of the invention, and other embodiments should be understood to be encompassed in the present disclosure as would be understood by those of ordinary skill in the art. All references cited hereinbefore are hereby incorporated by references in their entirety.

1. A topical composition comprising:
(a) a water soluble extract of an Uncaria species in a concentration range from about 0.1% to about 15% (w/w); and
(b) a dermatologically acceptable medium suitable for topical application.

2. The topical composition of claim 1, wherein the water soluble extract of an Uncaria species is in a concentration range from about 0.5% to 8% (w/w).

3. A method of improving skin cell's resistance to DNA damage and/or enhancing skin cell's DNA repair capacity comprising topically applying an effective amount of the topical composition of claim 1 on the skin.

4. A method of protecting skin from damage caused by exposure to ultraviolet light comprising topically applying an effective amount of the topical composition of claim 1 on the skin, prior to exposure to the ultraviolet light, to protect the skin from damage.

5. A method of treating skin damage caused by exposure to ultraviolet light comprising topically applying an effective amount of the topical composition of claim 1 on an area of the skin, after exposure of the area to the ultraviolet light, to treat the skin damage caused by the ultraviolet light.

6. A method of treating and/or preventing skin wrinkles, keratosis or lipofuscin deposit comprising topically applying an effective amount of the topical composition of claim 1 on the skin, to reduce and/or prevent the skin wrinkles, keratosis or lipofuscin deposit.

7. A topical composition comprising:
(a) a water soluble extract of an Uncaria species in a concentration range from about 0.1% to about 15% (w/w);
(b) deuterium-reduced water; and
(c) dermatologically acceptable carrier materials.

8. The topical composition of claim 7, wherein the deuterium reduced water has a deuterium concentration in a range from 0.1 ppm to about 110 ppm.

9. The topical composition of claim 7, wherein water soluble extract of an Uncaria species is in a concentration range from about 0.5% to 8% (w/w).

10. A method of improving skin cell's resistance to DNA damage and/or enhancing skin cell's DNA repair capacity comprising topically applying an effective amount of the topical composition of claim 7 on the skin.

11. A method of protecting skin from damage caused by exposure to ultraviolet light comprising topically applying an effective amount of the topical composition of claim 7 on the skin, prior to exposure to the ultraviolet light, to protect the skin from damage.

12. A method of treating skin damage caused by exposure to ultraviolet light comprising topically applying an effective amount of the topical composition of claim 7 on an area of the skin after exposure of the area to the ultraviolet light, to treat the skin damage caused by the ultraviolet light.

13. A method of treating and/or preventing skin wrinkles, keratosis or lipofuscin deposit comprising topically applying an effective amount of the topical composition of claim 7 on the skin, to reduce and/or prevent the skin wrinkles, keratosis or lipofuscin deposit.

14. A topical composition comprising:
(a) a water soluble extract of an Uncaria species in an amount sufficient to prevent and/or treat skin damage, caused from exposure to ultraviolet light; and
(b) a dermatologically acceptable medium.

15. The topical composition of claim 14 further comprising deuterium-reduced water.

16. The topical composition of claim 14, wherein the deuterium-reduced water has a deuterium concentration in a range from 0.1 ppm to about 110 ppm.
